(19) United States
(12) Patent Application Publication
(10) Pub. No.: US 2026/0110675 A1
(43) Pub. Date: Apr. 23, 2026

(54) HYDROGEN GAS MEASUREMENT SENSOR COMPRISING A GAP AND METHOD FOR MANUFACTURING THE SAME

(71) Applicant: INDUSTRY-ACADEMIC COOPERATION FOUNDATION, YONSEI UNIVERSITY, Seoul (KR)

(72) Inventors: Woo Young LEE, Seoul (KR); Sang Kil LEE, Seoul (KR); Joon Ho LEE, Seoul (KR); Ji Woo HONG, Seoul (KR)

(21) Appl. No.: 19/364,010

(22) Filed: Oct. 21, 2025

(30) Foreign Application Priority Data
Oct. 22, 2024 (KR) 10-2024-0144589
Aug. 20, 2025 (KR) 10-2025-0116070

(51) Int. Cl. G01N 33/00; G01N 27/12

(52) U.S. Cl. CPC G01N 33/005; G01N 27/125

(57) ABSTRACT

An embodiment relates to a hydrogen gas sensor for forming an electrically separated nanogap in a Pd thin film through an ultrasonic treatment and hydrogen cycling, wherein the formed nanogap opens and closes according to a hydrogen concentration, enabling quantitative and sensitive detection through a change in capacitance, and has advantages in that a uniform gap structure can be implemented with a simple process without mechanical straining, providing excellent repeatability and manufacturing reproducibility, and that the sensitivity and detection limit of the sensor can be precisely controlled only with the ultrasonic treatment conditions.

### CROSS REFERENCE TO RELATED APPLICATION

The present application claims priority to Korean Patent Application No. 10-2024-0144589, filed on Oct. 22, 2024, and Korean Patent Application No. 10-2025-0116070, filed on Aug. 20, 2025, and the entire contents of which are incorporated here for all purposes by this reference.

### BACKGROUND

### 1. Technical Field

The present invention relates to a hydrogen gas measurement sensor comprising a gap induced by ultrasonic treatment and a method for manufacturing the same.

### 2. Related Art

Recently, as hydrogen is drawing attention as a clean energy, its utilization in various industrial fields such as hydrogen vehicles, fuel cells, and hydrogen storage systems is rapidly increasing. Accordingly, the demand for high-performance hydrogen sensors is also increasing throughout the entire life cycle ranging from hydrogen production, storage, transportation, and utilization. In particular, hydrogen is a colorless, odorless, and flammable gas and has a high risk of explosion upon leakage, and thus, it is essential to secure a high-sensitivity sensor technology capable of detecting whether there is a leak in real-time.

Conventional hydrogen sensor technologies have been mainstreamed by metal oxide-based resistive-type sensors or nanogap sensors in which cracks are induced in a Pd thin film by a mechanical stretching method, but these sensors have a limitation in that they have high operating temperatures, or have complex manufacturing processes and low reproducibility. In particular, in the case of Pd nanogap sensors, because the crack formation method relies on mechanical stretching, or it is difficult to precisely control the crack size and density, there have been limitations to commercialization in terms of sensitivity and repeatability.

Therefore, the development of a new concept of hydrogen sensor technology, which has a simple manufacturing process, can form a uniform nanogap structure, enables hydrogen detection even at room temperature, and can output quantitative and repeatable electrical signals, is urgently needed.

### SUMMARY

The present invention has been devised to solve the above-mentioned problems, and one embodiment of the present invention provides a hydrogen gas measurement sensor.

In addition, another embodiment of the present invention provides a method for manufacturing a hydrogen gas measurement sensor.

The technical problems to be solved by the present invention are not limited to the technical problems mentioned above, and other unmentioned technical problems will be clearly understood by a person having ordinary skill in the art to which the present invention pertains from the following description.

As a technical means for achieving the above-mentioned technical problems, one aspect of the present invention provides the following:

A hydrogen gas measurement sensor, comprising: an insulating substrate; an oxide layer stacked on the insulating substrate; a rubber substrate stacked on the oxide layer; and a catalyst layer stacked on the rubber substrate; wherein the catalyst layer comprises at least one gap located on a surface or inside thereof.

The insulating substrate may comprise a doped silicon (Si) substrate.

The oxide layer may comprise silicon oxide (SiO₂).

The rubber substrate may comprise polydimethylsiloxane (PDMS).

The catalyst layer may comprise at least one selected from the group consisting of palladium (Pd), platinum (Pt), aluminum (Al), nickel (Ni), manganese (Mn), molybdenum (Mo), magnesium (Mg), vanadium (V), and combinations thereof.

The gap may be an electrical disconnection structure (nanogap) operably formed through a hydrogenation and dehydrogenation process of a microcrack induced by ultrasonic treatment.

The gap may have a width of 10 to 50 nm.

An interval between the gaps may be 0.01 to 5 μm.

Even after repeating adsorption and desorption of hydrogen 50 times or more under a condition of a hydrogen gas concentration of 300 ppm, an opening and closing behavior and a capacitance change of the gap may be maintained in a range of 90% to 110% of a reference value.

Another aspect of the present invention provides the following:

A method for manufacturing a hydrogen gas measurement sensor, comprising the steps of: (a) forming an insulating substrate; (b) forming an oxide layer on the insulating substrate; (c) forming a rubber substrate on the oxide layer; (d) forming a catalyst layer on the rubber substrate; and (e) performing an ultrasonic treatment on the catalyst layer to form at least one gap on a surface or inside of the catalyst layer.

The ultrasonic treatment may be performed for 5 seconds to 5 minutes in an ethanol solvent.

The gap may be an electrical disconnection structure (nanogap) operably formed through a hydrogenation and dehydrogenation process of a microcrack induced by ultrasonic treatment.

The method for manufacturing may further comprise the step of: (f) performing a hydrogen gas (H₂) treatment.

The hydrogen gas may have a concentration of 1 to 10 vol %.

The step (f) may be performed by exposure to hydrogen gas for 1 minute to 5 minutes.

According to one embodiment of the present invention, because the hydrogen gas measurement sensor can simply and reproducibly form an operable nanogap only by sonication and hydrogen treatment, a high-sensitivity sensor can be manufactured even without complex mechanical equipment or expensive stretching systems, so that the manufacturing process may not only be economical but may also contribute significantly to the environment.

In addition, according to one embodiment of the present invention, because the capacitance (C) of the nanogap-based sensor changes continuously according to a change in a contact area between Pd islands, and a response signal can be quantitatively output according to a hydrogen concentration, it has an advantage of being able to provide excellent performance in terms of sensitivity, selectivity, and reproducibility compared to conventional resistive-type (on/off) sensors.

Furthermore, according to one embodiment of the present invention, by controlling the ultrasonic treatment time and the hydrogen treatment conditions, the size, interval, and density of the gap can be precisely controlled, and through this, the sensor's minimum detection concentration (LOD), response speed, saturation concentration, etc., can be custom-adjusted, so that it has an advantage of being able to be effectively applied to hydrogen leakage detection and real-time monitoring applications in various industrial fields.

The effects of the present invention are not limited to the effects described above, and should be understood to include all effects that can be inferred from the configurations of the invention described in the detailed description of the invention or the claims.

### BRIEF DESCRIPTION OF THE DRAWINGS

FIG. 1 is a schematic diagram illustrating an operating structure of a sensor and a concept of capacitance change according to hydrogen adsorption according to one embodiment of the present invention.

FIG. 2 is a diagram illustrating a sensor manufacturing process according to one embodiment of the present invention.

FIG. 3 is a block diagram illustrating a manufacturing process, a sensing mechanism, and a change in an effective area of an electrode according to a hydrogen concentration of a sensor according to one embodiment of the present invention.

FIG. 4 is a diagram structurally comparing nanogap formation methods of Comparative Example 1 (CPE), Comparative Examples 2 and 3 (MOTIFE), and Example 1 (SANG) according to the present invention.

FIG. 5 is an image showing a process in which a nanogap is formed in a sensor according to one embodiment of the present invention, observed step-by-step with an optical microscope and an electron microscope.

FIG. 6 is an analysis result comparing changes in nanogap size, crack density, and hydrogen sensitivity (Limit of Detection) according to ultrasonic treatment conditions according to one embodiment of the present invention.

FIG. 7 is a graph showing hydrogen sensing characteristics, response speed, repeatability, and selectivity of a sensor according to one embodiment of the present invention.

### DETAILED DESCRIPTION

Hereinafter, the present invention will be described in more detail. However, the present invention may be embodied in many different forms, is not limited by the embodiments described herein, and is only defined by the claims that will be described later.

In addition, the terminology used in the present invention is for the purpose of describing particular embodiments only and is not intended to be limiting of the present invention. The singular forms include the plural forms unless the context clearly dictates otherwise. Throughout the specification of the present invention, stating that a certain component is ‘included’ means that other components may be further included, rather than excluding other components, unless there is a specific statement to the contrary.

Throughout the specification, when a part is referred to as being “connected (connected, in contact, coupled)” to another part, this includes not only cases of being “directly connected” but also cases of being “indirectly connected” with another member interposed therebetween. In addition, when a part is said to “comprise” a certain component, this means that it may further be provided with other components, rather than excluding other components, unless there is a specific statement to the contrary.

The “slurry composition” as used herein may mean a slurry composition for polishing a target film material, and the polishing may mean Chemical Mechanical Polishing (CMP).

The “%” as used herein, unless otherwise specified, may mean “% by weight” or “wt %” in terms of content.

The terminology used herein is for the purpose of describing particular embodiments only and is not intended to be limiting of the present invention. The singular forms include the plural forms unless the context clearly dictates otherwise.

A first aspect of the present invention provides:

A hydrogen gas measurement sensor, comprising: an insulating substrate; an oxide layer stacked on the insulating substrate; a rubber substrate stacked on the oxide layer; and a catalyst layer stacked on the rubber substrate; wherein the catalyst layer comprises at least one gap located on a surface or inside thereof.

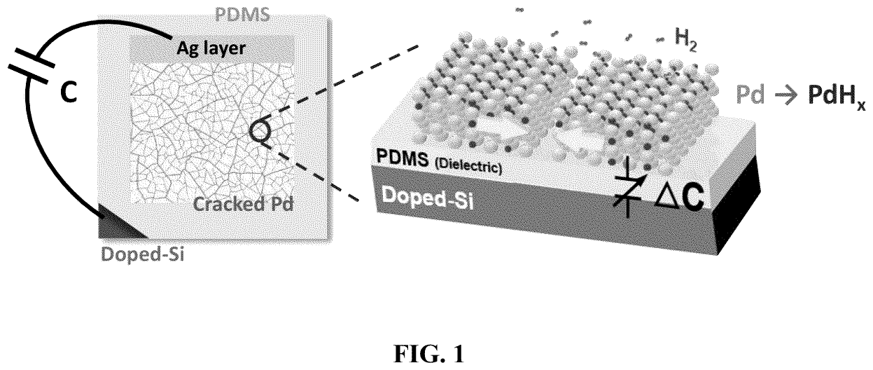

Hereinafter, the hydrogen gas measurement sensor according to the first aspect of the present invention will be described in detail with reference to FIG. 1.

In one embodiment of the present invention, FIG. 1 is a schematic diagram schematically illustrating the structure and hydrogen sensing principle of the hydrogen measurement sensor according to the present invention. Referring to FIG. 1, the sensor may have a structure in which a PDMS dielectric layer is formed on a doped silicon substrate (Doped-Si), and a Pd catalyst layer having a cracked structure is stacked thereon. Pd adsorbs hydrogen and undergoes a phase transition to PdHx, inducing lattice expansion, and due to this, as the gap between the Pd islands closes, an effective contact area with an upper Ag electrode may increase. Accordingly, the capacitance increases according to the relationship C∝A/d, and the sensor can generate a quantitative response signal directly proportional to the hydrogen concentration based on this change in electrode area.

In one embodiment of the present invention, the insulating substrate may be a doped silicon substrate.

In one embodiment of the present invention, the insulating substrate acts as an electrode together with the catalyst layer, and serves as a lower electrode during capacitance measurement.

In one embodiment of the present invention, the insulating substrate and the catalyst layer may be connected by a silver paste (Ag paste) to achieve electrical connection.

In one embodiment of the present invention, an oxide layer may be formed on the insulating substrate.

In one embodiment of the present invention, the oxide layer may comprise silicon oxide (SiO₂).

In one embodiment of the present invention, the oxide layer may be characterized in that it imparts insulating and dielectric properties, and at the same time, provides improved adhesion with the rubber substrate (PDMS) and structural stability.

In one embodiment of the present invention, a rubber substrate may be formed on the oxide layer.

In one embodiment of the present invention, the rubber substrate may be polydimethylsiloxane (PDMS).

In one embodiment of the present invention, the PDMS is a rubber material having elasticity against external stress, serves as a dielectric, and enables stress dispersion and strain absorption for expansion/contraction of the catalyst layer according to hydrogen adsorption.

In one embodiment of the present invention, the PDMS is formed into a thin film and coated on the doped silicon substrate, acts as a dielectric within the hydrogen detection sensor, and is interposed between two electrodes during capacitance measurement.

In one embodiment of the present invention, a catalyst layer may be formed on the rubber substrate.

In one embodiment of the present invention, the catalyst layer is stacked via the oxide layer and the rubber substrate formed on the doped silicon substrate, and acts as a main sensing part for hydrogen detection.

In one embodiment of the present invention, the catalyst layer may comprise at least one selected from the group consisting of palladium (Pd), platinum (Pt), aluminum (Al), nickel (Ni), manganese (Mn), molybdenum (Mo), magnesium (Mg), vanadium (V), and combinations thereof, and for example, may be formed of a thin film comprising palladium (Pd), and may be deposited on the surface of the PDMS rubber substrate to a thickness of about 10 nm.

In one embodiment of the present invention, a discontinuous microcrack (seed crack) is induced in the catalyst layer through an ultrasonic (sonication) treatment, and thereafter, as the Pd thin film adsorbs hydrogen and undergoes a phase transition to PdHx in a 4 vol % hydrogen atmosphere, stress relief and separation between Pd fragments occur, thereby forming an electrically isolated structural gap (nanogap). The gap exhibits a dynamic behavior of closing or opening according to the hydrogen concentration, which changes the effective area of the upper electrode, leading to an increase or decrease in capacitance.

In one embodiment of the present invention, unlike conventional tension-based nanogap technology, the gap formed in the present invention can be simply formed only by ultrasonic treatment without separate mechanical equipment, and by quantitatively controlling the seed crack formation step and the hydrogenation step, the position, size, density, and uniformity of the gap can be secured at a high level. As a result, the deviation in characteristics between devices can be minimized, and excellent reproducibility and reliability can be provided in terms of repeated sensing characteristics and signal stability compared to conventional technology.

In one embodiment of the present invention, the nanogap is structurally random, but the width and interval of the gap can be critically controlled according to process conditions such as the ultrasonic treatment time, and this acts as a decisive factor in setting the hydrogen detection sensitivity and detection range. Specifically, if the average width of the gap is less than 10 nm, unintended conductive paths may be formed due to excessively frequent contact between Pd islands, and conversely, if it exceeds 50 nm, it becomes difficult for the gap to close according to changes in hydrogen concentration, which can cause a sharp drop in detection sensitivity. Therefore, setting the gap width to 10-50 nm corresponds to a technical critical condition for securing the sensitivity and operational stability of the sensor.

In one embodiment of the present invention, when the interval between the gaps is formed in a range of 0.01-5 μm, the Pd islands are arranged at appropriate intervals to selectively induce contact/non-contact according to changes in hydrogen concentration, thereby enabling the generation of a quantitative, capacitance-based detection signal. In a narrower interval, signal distortion may occur due to electron tunneling or unintended contact between the gaps, and conversely, if the interval is too wide, the number of effective gaps that react to hydrogen within the entire Pd layer is significantly reduced, which may degrade the reactivity of the sensor.

In one embodiment of the present invention, the catalyst layer may be composed of a plurality of randomly distributed Pd islands, and the average width of the nanogaps formed between them may be in the range of 10 to 50 nm, and the interval between the gaps may be in the range of 0.01 to 5 μm. Since the density and size of the gaps can be quantitatively controlled according to the ultrasonic treatment time, it has the advantage of being able to customize the hydrogen detection sensitivity and detection range.

In one embodiment of the present invention, even after repeating the adsorption and desorption of hydrogen 50 times or more under a condition of a hydrogen gas concentration of 300 ppm, the opening and closing behavior of the gap and the corresponding change in capacitance may be maintained within ±10%.

In one embodiment of the present invention, because the gap is formed by quantitatively controlling the ultrasonic treatment conditions and the initial hydrogenation conditions, uniform structural characteristics are secured even among devices manufactured in the same process, and the contact/non-contact operation between Pd islands can be stably maintained even in an environment of repeated exposure to hydrogen gas.

This repeated sensing stability is distinguished from the problem of decreased repeatability that occurs in conventional resistive-type sensors due to deviations in crack position, non-uniformity of crack size, and differences in characteristics between devices, and in particular, the ability to maintain a repeated sensing signal within a quantitative deviation range of ±10% can act as a critical structural/operational stability standard for minimizing performance degradation in real-time monitoring, ensuring environmental stability, and during long-term operation of hydrogen gas detection sensors.

In one embodiment of the present invention, an Ag layer may be selectively applied to a partial region of the catalyst layer, and the Ag layer may define a minimum effective area of an upper electrode and induce a change in the capacitance signal by causing the Pd islands to contact the Ag layer upon hydrogen adsorption.

Consequently, the catalyst layer of the present invention can function as a high-sensitivity and high-reproducibility capacitance-based hydrogen detection sensor by integrating the lattice expansion characteristic of Pd due to hydrogenation and the crack structure induced by the ultrasonic treatment. The gap acts as a structural sensing element that is very advantageous for detecting quantitative changes in hydrogen concentration by inducing area closure and capacitance increase in proportion to the hydrogen concentration.

A second aspect of the present invention provides:

A method for manufacturing a hydrogen gas measurement sensor, comprising the steps of: (a) forming an insulating substrate; (b) forming an oxide layer on the insulating substrate; (c) forming a rubber substrate on the oxide layer; (d) forming a catalyst layer on the rubber substrate; and (e) performing an ultrasonic treatment on the catalyst layer to form at least one gap on a surface or inside of the catalyst layer.

Detailed descriptions of features overlapping with the first aspect of the present invention are omitted, but it is understood that the descriptions for the first aspect of the present invention may be applied equally to the second aspect, even where such descriptions have been omitted.

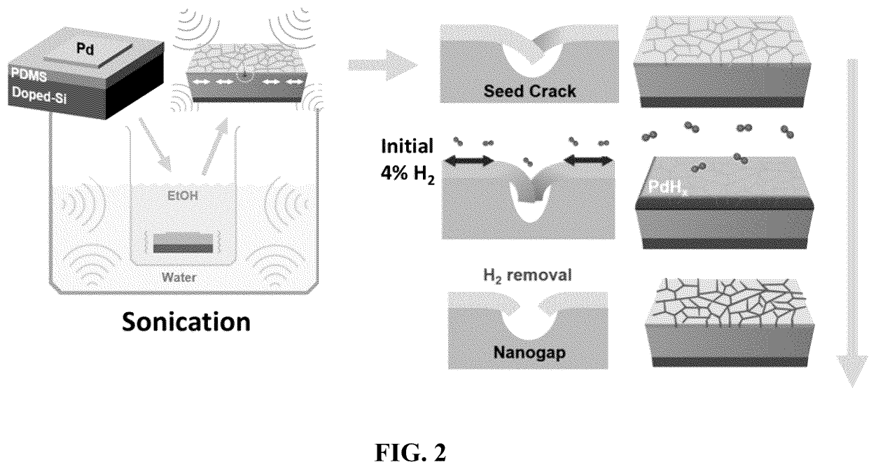

Hereinafter, the method for manufacturing a hydrogen gas measurement sensor according to the second aspect of the present invention will be described in detail with reference to FIG. 2.

In one embodiment of the present invention, the hydrogen gas measurement sensor may be manufactured through the following process steps.

In one embodiment of the present invention, a step of (a) forming an insulating substrate may be included, and the insulating substrate is preferably composed of a doped silicon wafer.

In one embodiment of the present invention, (b) an oxide layer composed of SiO₂ or a similar insulating material may be formed on the insulating substrate.

In one embodiment of the present invention, (c) a rubber substrate composed of polydimethylsiloxane (PDMS) may be formed on the oxide layer.

In one embodiment of the present invention, a step of (d) forming a catalyst layer comprising palladium (Pd) or other catalytic metals on the rubber substrate may be included.

In one embodiment of the present invention, the catalyst layer is preferably formed as a thin film with a thickness of about 10 nm by a sputtering deposition method, and to ensure sensing characteristics, may be designed in a continuous film or a patterned structure.

In one embodiment of the present invention, the method includes a step of (e) performing an ultrasonic treatment (sonication) on the catalyst layer to form at least one gap on a surface or inside of the catalyst layer.

In one embodiment of the present invention, the ultrasonic treatment may be performed in an ethanol solvent for a time of 5 seconds or more and 5 minutes or less, and this range acts as a critical condition for forming uniform and dense seed cracks in the Pd thin film.

In one embodiment of the present invention, with a short treatment time of less than 5 seconds, the generation of cracks is insufficient, making it difficult to operate as a sensing structure, while an excessive treatment exceeding 5 minutes may increase the possibility of detachment or unintentional peeling of the Pd thin film. The seed crack induced through such optimal ultrasonic conditions develops into a random yet controllable nanogap between the Pd islands, and serves as a main structure that determines the sensitivity and reproducibility of the hydrogen detection performance.

In one embodiment of the present invention, after the ultrasonic treatment, a step of (f) treating with hydrogen gas may be further included.

In one embodiment of the present invention, the hydrogen gas preferably has a concentration in the range of 1 vol % to 10 vol %, and more specifically, may be exposed for 1 minute to 5 minutes under a condition of 2 to 6 vol %.

In one embodiment of the present invention, the above range corresponds to the concentration and time conditions that can cause the Pd lattice to undergo a phase transition from α-PdH to β-PdH and a lattice expansion of about 3.5%, which is a crucial step for the seed crack to develop into an electrically completely isolated structure and convert into an actually operable gap.

In one embodiment of the present invention, if the concentration of the hydrogen treatment condition is less than 1 vol %, the phase transition of the Pd lattice may be insufficient, leading to a possibility that the gap may not close or may fail to act as a sensing structure, and if it exceeds 10 vol %, problems such as mechanical delamination or electrode damage may occur due to excessive expansion of the Pd layer.

In one embodiment of the present invention, in terms of time, less than 1 minute is not sufficient to induce the phase transition, and an excessive exposure exceeding 5 minutes may degrade the elastic recovery of the gap due to the excessive formation of PdHx.

In one embodiment of the present invention, the hydrogen detection sensor formed through the manufacturing method as described above may have the following technical advantages over conventional technologies.

First, conventional nanogap-based hydrogen sensors used a method of forming cracks by applying a repetitive mechanical straining and releasing process to a Pd thin film formed on a PDMS substrate, and as a result, the direction of the cracks was not uniform, and the interval and position of the gaps were irregularly formed, which caused problems of deviation between sensors and decreased repeatability. Furthermore, the process essentially required mechanical straining equipment, had low process reproducibility, and had limitations in securing productivity.

In contrast, the manufacturing method of the present invention can form uniform and multi-directional seed cracks in the Pd thin film with only a simple ultrasonic treatment process without a separate mechanical device.

Because the density and size of the gaps can be quantitatively controlled simply by adjusting the ultrasonic treatment time, structural uniformity between devices and the reproducibility of sensing characteristics can be effectively ensured.

In addition, the concentration and time conditions set in the hydrogen treatment step induce a stable phase transition of the Pd lattice, transforming the seed crack from a simple crack state into an electrically disconnected, operable gap (nanogap), which allows it to function as a sensing structure.

Moreover, unlike conventional resistance-based (on/off) sensors, the present invention can generate a quantitative signal corresponding to the hydrogen concentration through a change in capacitance based on the continuous change in the effective area of the electrode according to the opening and closing of the gap, which is advantageous in terms of sensitivity and resolution.

In one embodiment of the present invention, FIG. 2 is a schematic diagram illustrating the manufacturing process of the hydrogen gas measurement sensor according to the present invention. Referring to FIG. 2, after an oxide layer and a PDMS rubber substrate are formed on a doped silicon substrate, a Pd thin film is deposited, and a seed crack is induced through an ultrasonic treatment in an ethanol solvent. Subsequently, by treating with 4 vol % hydrogen gas, a PdHx phase transition is induced, and due to stress relief from lattice expansion, the seed crack is transformed into an operable electrical disconnection structure (nanogap). Such a process flow is the core manufacturing method of the present invention, which can simply manufacture an operable nanogap structure without complex straining equipment.

Consequently, the manufacturing method of the present invention can implement a hydrogen detection sensor that surpasses conventional technology in terms of process simplicity, structural controllability, repeated sensing stability, and ensuring signal quantifiability.

Hereinafter, embodiments of the present invention will be described in detail so that a person having ordinary skill in the art to which the present invention pertains can easily carry it out. However, the present invention may be embodied in many different forms and is not limited to the embodiments described herein.

### Preparation Example 1: Manufacture of Hydrogen Detection Sensor

### Example 1 (SANG)

In a preparation example of the present invention, a PDMS rubber substrate was formed on a doped silicon substrate, and after a Pd thin film was deposited thereon, a hydrogen sensor having a nanogap formed therein was manufactured through an ultrasonic treatment and a hydrogen gas treatment process.

First, a 4-inch B-doped silicon wafer was used as a substrate, and an SiO₂ oxide film with a thickness of about 2000 Å was formed on its surface. The elastomer, PDMS, was prepared by mixing Sylgard 184 (Dow Corning) and a curing agent at a weight ratio of 10:1, to which hexane (purity ≥95%) was then added and mixed at a 3:1 weight ratio, and the mixture was subjected to ultrasonic treatment for 30 minutes. Subsequently, after treating the wafer surface with a 100 W O₂ plasma for 3 minutes to improve the interfacial adhesion with the PDMS, the PDMS mixture was spin-coated at 3000 rpm for 30 seconds and cured at 80° C. for 6 hours to form a PDMS layer with a thickness of about 11 μm. The cured wafer was cut into a size of 1.8×1.8 cm² and pretreated through acetone ultrasonic cleaning (10 minutes), an IPA rinse, and N₂ gas drying.

Next, a palladium (Pd) thin film with a thickness of 10 nm was deposited on the PDMS substrate. The deposition was performed by an ultra-high vacuum DC magnetron sputtering method, under conditions of a base pressure of 5×10⁻⁸ Torr, a pressure of 2.4×10⁻³ Torr or less in an argon atmosphere, and a flow rate of 34 sccm. The deposition was carried out at a deposition rate of 3.5 Å/s, and a Pd target with a purity of 99.9% was used. The deposition area was limited to a 1×1 cm² region using a PI tape mask.

On the Pd thin film, an ultrasonic treatment (POWERSONIC 405, HWASHIN) was performed in an ethanol (EtOH) solvent to form a seed crack. The ultrasonic treatment was carried out at maximum power for a time between 5 seconds and 5 minutes, and this is a pre-structural induction process for the subsequent formation of nanogaps according to the hydrogen reaction. Subsequently, a 50 nm thick silver (Ag) layer was sputtered on some regions to secure a reference contact area for the upper electrode.

After the ultrasonic treatment, to induce the formation of nanogaps, the sample was exposed for 200 seconds under a condition of 4 vol % hydrogen gas (N₂ balance), and was then converted to a state having a stable reference capacitance (C₀) by repeating a purging process with N₂ several times. Through this cycle, the crack induced by the ultrasonic treatment developed into an electrically separated nanogap according to the lattice expansion and stress accumulation from the PdHx phase transition, thereby manufacturing Example 1.

Example 1 and Comparative Examples 1-3 are as follows.

| | | | | | Gap | |
|---|---|---|---|---|---|---|
| | Nanogap formation | | | Measurement | Size | LOD |
| | method | | Substrate | Mechanism | (nm) | (ppm) |
| Comparative | CPE (4% H₂ to uncracked | | PDMS | Resistive | 900 | 4,000 |
| Example 1 | Pd) | | | | | |
| Comparative | MOTIFE | 60%-strained | | | 254 | 2000 |
| Example 2 | (Stretching) | Pd | | | | |
| Comparative | | 120%-strained | | | 94 | 50 |
| Example 3 | | Pd | | | | |

| | | | | | |
|---|---|---|---|---|---|
| Example 1 | SANG | PDMS/Si | Capacitive | [symbol] 20 | 3 |
| | (Sonication-assisted Pd | | | | |
| | Nanogap) | | | | |

Comparative Example 1: J. Lee, J. S. Noh, S. H. Lee, B. Song, H. Jung, W. Kim, W. Lee, Int. J. Hydrogen Energy 2012, 37, 7934-7939.
Comparative Example 2, 3: S. Park, S.-M. Lee, J.-K. Jeong, D. Kim, H. Kim, H.-S. Lee, W. Lee, Sens. Actuators B: Chem., 2021, 348, 130716

### Experimental Example 1: Analysis of Sensor Operating Structure and Principle of Capacitance Change

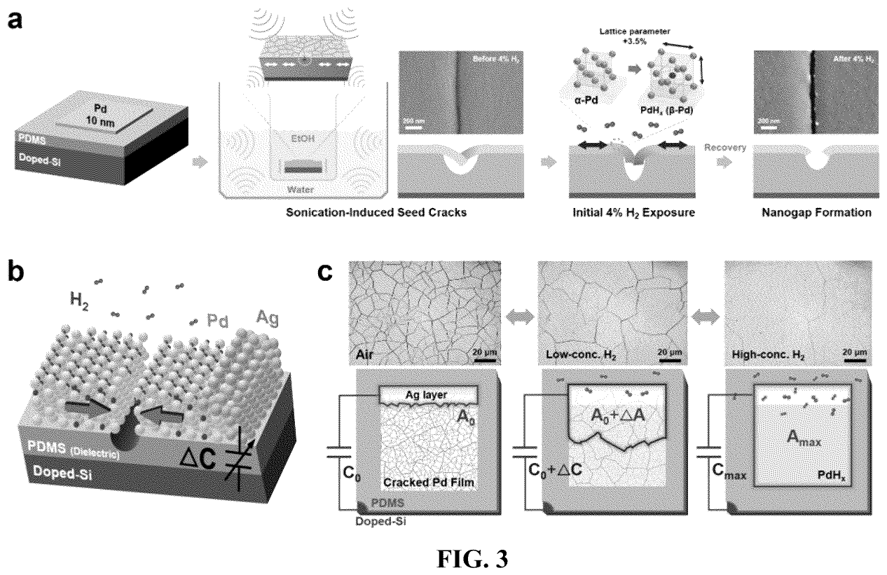

FIG. 3 visually illustrates the structure, operating mechanism, and principle of capacitance change of the hydrogen detection sensor based on Example 1 (SANG sensor).

FIG. 3a is a conceptual diagram of the process by which the sensor of the present invention is manufactured. A 10 nm thick Pd thin film was formed on a spin-coated, doped silicon substrate with PDMS, and a seed crack was induced through an ultrasonic treatment in an ethanol medium. Subsequently, in a 4% hydrogen atmosphere, the Pd thin film underwent a phase transition to PdHx and a lattice expansion of about 3.5% occurred, which caused the cracks in the Pd thin film to expand and an electrically isolated nanogap to be formed. Actual SEM images before and after the nanogap formation are also presented, and it is confirmed that the initially smooth surface shows clear cracks after the hydrogen cycle.

FIG. 3b illustrates the sensing mechanism of the sensor. Hydrogen (H₂) molecules are adsorbed onto the Pd thin film to form PdHx, which causes the gap to close as the distance (gap) between the Pd islands decreases. When the Pd islands come into contact with the upper Ag electrode, the effective electrode area (A) increases, and accordingly, the capacitance (C) increases according to the relationship C=εₒεᵣA/d. This illustrates that the phase transition and volume expansion of Pd serve as the physical basis for inducing the Pd—Ag electrical connection, which in turn generates ΔC.

FIG. 3c quantitatively represents the change in the effective electrode area (A) and capacitance (C) under conditions of air (no hydrogen), low-concentration hydrogen, and high-concentration hydrogen. In air, the Pd area connected to the upper Ag electrode corresponds to Aₒ, and the capacitance at this time is the reference capacitance (Cₒ). When hydrogen is introduced at a low concentration, some Pd islands become connected, and the electrode area increases to Aₒ+ΔA, and the capacitance rises to Cₒ+ΔC. Under high-concentration hydrogen, most of the Pd islands contact the Ag, the effective area reaches Amax, and the capacitance increases to Cₘₐₓ. This illustrates that the present invention has a structure capable of outputting an analog sensing signal directly proportional to the hydrogen concentration by detecting the change in capacitance based on such a change in the effective area.

Through this example, it was confirmed that the ultrasonic-induced nanogap structure based on Pd/PDMS can induce a quantitative capacitance change according to a change in hydrogen concentration, which demonstrates the technical superiority of the present invention, differentiating it from the conventional resistance-based (on/off) method.

### Experimental Example 2: Comparative Analysis of Nanogap Formation Methods

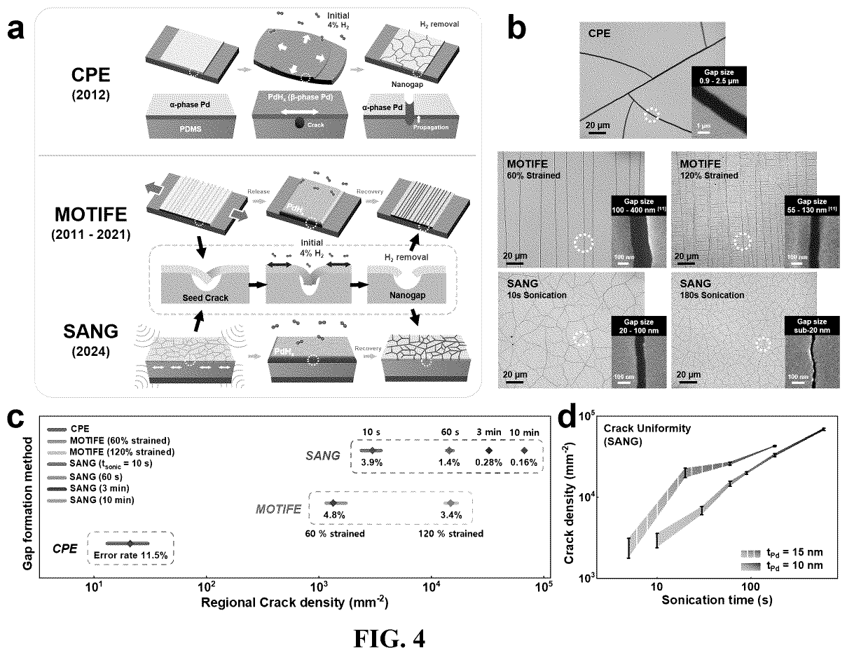

FIG. 4 compares the characteristics of SANG with previously established nanogap formation techniques, all of which involve inducing a phase transition in Pd by exposing it to an initial high concentration of H₂ (FIG. 4a). In this study, we quantified the crack density-defined as the number of Pd fragments per unit area-allowing for a direct comparison of the different nanogap formation methods. Crack density was determined by counting the average number of intersections between the cracks and the horizontal and vertical grids observed in a 100× magnified OM image. For cracked Pd films on elastomeric substrates (CPE), cracks first form at the Pd/PDMS

interface due to tensile stress from the areal expansion of Pd during the initial H₂ exposure. After dehydrogenation, as Pd reverts to the a-phase, these cracks penetrate through the Pd thin film, resulting in gaps as wide as 900 nm (FIG. 4b).

For highly mobile thin films on elastomers (MOTIFE), repeated cycles of mechanical stretching and release create parallel seed cracks perpendicular to the strain direction in the Pd/PDMS system. These cracks evolve into operational nanogaps upon initial H₂ exposure through a mechanism similar to SANG.

For SANG, extending the sonication time (t_{sonic}) for the Pd/PDMS/Si system resulted in an increased density of seed cracks and smaller gap sizes after the initial H₂ exposure. When t_{sonic} exceeded 3 min, SANGs exhibited extremely fine, uniformly distributed gaps across the Pd surface, which were notably smaller than those produced by the CPE and MOTIFE methods. Scanning electron microscopy (SEM) revealed that SANGs created with t_{sonic} longer than 3 min were narrower than 20 nm, which represents a significant reduction compared to previously reported nanogaps for H₂ sensors.

FIG. 4c compares the average crack density and associated error across different areas for the three nanogap formation methods. The CPE method, with the lowest average crack density, exhibited the highest error across different regions, indicating an uneven distribution of nanogaps. In the MOTIFE method (with 60% and 120% strained, respectively), as the tensile strain increased, crack density increased while the error decreased. For SANG, when the sonication time exceeded 1 min, the crack density surpassed that of 120% strained MOTIFE, while exhibiting significantly lower error rates.

FIG. 4d shows the crack density curves for SANGs with different Pd thicknesses (t_{pd}=10 nm, 15 nm) as a function of t_{sonic}. In both cases, error across different regions decreased with increasing t_{sonic}, leading to more uniform nanogaps.

### Experimental Example 3: Observation of Structural Changes in Each Stage of Nanogap Formation

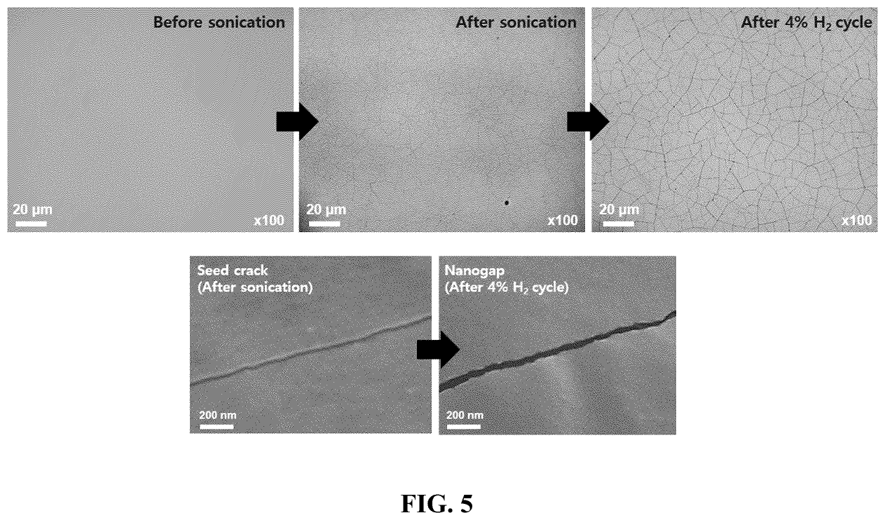

FIG. 5 shows the results of visually observing the series of structural changes in which a nanogap is formed on the surface of the Pd thin film during the manufacturing process of the SANG-based hydrogen gas measurement sensor. In particular, the states before/after the ultrasonic treatment and after a 4% hydrogen cycle were compared step-by-step using an optical microscope (OM) and a scanning electron microscope (SEM).

The upper part of FIG. 5 shows the surface of the Pd thin film at three process stages, taken with an optical microscope (×100 magnification), sequentially showing the states from the left: before sonication, after sonication, and after a 4% H₂ cycle.

Before sonication, a smooth and uniform surface without cracks is observed, and after the ultrasonic treatment, uniformly distributed microcracks (seed cracks) are formed over the entire surface.

Subsequently, after undergoing a 4% hydrogen treatment and dehydrogenation cycle, these seed cracks expand with clear definition and change into a state developed into a nanogap structure.

These optical images visually demonstrate that the nanogap is controllably formed according to the actual process conditions.

The lower part of FIG. 5 is an image of the same sample taken at high magnification (200 nm scale) with a scanning electron microscope (SEM).

The left image shows the initial state of the seed crack formed immediately after the ultrasonic treatment, in which fine and shallow linear cracks are observed on the surface.

The right image was taken at the same location after a 4% hydrogen cycle, showing the existing seed crack transformed into a completely separated electrical disconnection structure with depth and definition.

This experimentally shows that the PdHx phase transition and lattice expansion due to hydrogenation induce stress expansion, and that the gap thereby opens and grows into an operable nanogap.

Through this example, the structural mechanism by which a nanogap is formed in a Pd thin film through ultrasonic treatment and hydrogen cycling was visually confirmed, and the validity and reproducibility of the “seed crack→nanogap” transition process, which is the core concept of the present invention, were experimentally demonstrated.

### Experimental Example 4: Change in Nanogap Characteristics According to Ultrasonic Treatment Time

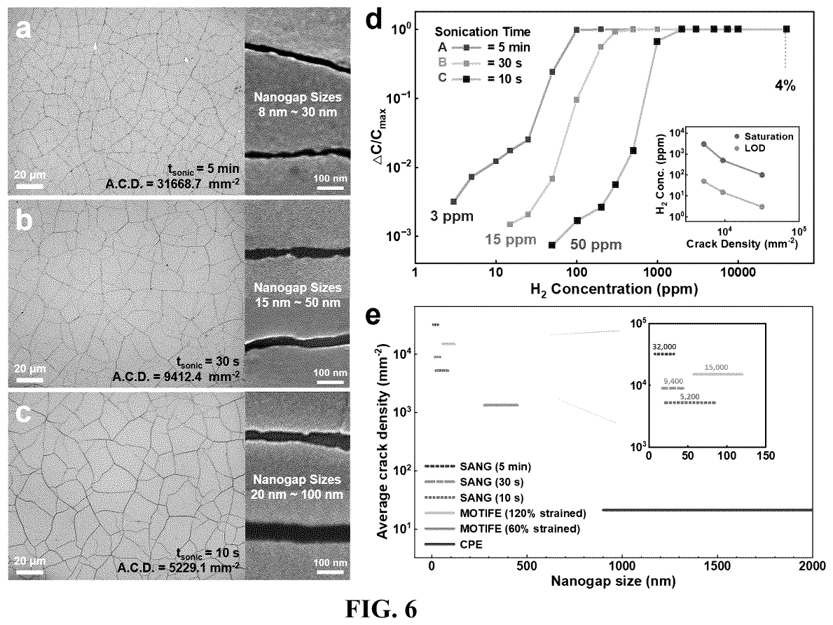

FIG. 6 shows how the structural characteristics of the nanogap and the hydrogen sensing characteristics are controlled according to the change in the ultrasonic treatment time (tₛₒₙᵢc) in the Example 1 sensor with the Pd/PDMS structure.

FIG. 6a-6c are images of the surface of the Pd thin film treated by sonication for 5 minutes, 30 seconds, and 10 seconds, respectively, taken with an optical microscope (OM) and a scanning electron microscope (SEM). As the ultrasonic treatment time increases, a trend is observed in which the crack density in the surface increases and the average gap size decreases. In particular, in the case of the 5-minute treatment, the size of the Pd islands was small and the gap size was precisely controlled to a level of 20 nm or less, whereas in the case of the 10-second treatment, the gap was relatively wide and exhibited poor uniformity.

FIG. 6d shows the results of quantitatively measuring the capacitance response (ΔC/ΔCₘₐₓ) over time according to the hydrogen concentration (3-500 ppm) for each sensor sample (A, B, C), which shows that the longer the ultrasonic treatment time, the more sensitively the sensor reacts even at low concentrations. The 5-minute sample reached a saturated response even at 3 ppm, while the 10-second sample showed a difference in that the response began only at 100 ppm or higher.

FIG. 6e is a two-dimensional diagram comparing the range of gap size and crack density distribution that can be achieved by the SANG process of the Example in comparison with the Comparative Examples of conventional nanogap formation methods (CPE, MOTIFE). It is visually confirmed that the manufacturing method of Example 1 enables both high-density cracks and precise gap formation simultaneously, and in particular, has the advantage of being able to form a uniform Pd island distribution with a multi-directional stress distribution caused by the ultrasonic treatment rather than directional straining.

In the case of the conventional MOTIFE method, a seed crack is formed by forming a metal thin film on a flexible substrate and then repeatedly applying mechanical straining in a perpendicular direction. However, since this method induces cracks in a single direction perpendicular to the straining direction, the distribution of the generated Pd islands and nanogaps is directional, which can lead to non-uniform sensing characteristics and spatial sensitivity deviation.

In fact, as a result of OM analysis, in the MOTIFE method, the crack density on the vertical axis with respect to the straining direction was high, but it was hardly observed on the horizontal axis, clearly showing the asymmetry of the 2D crack distribution.

On the other hand, the ultrasonic-based method of the present invention can secure a uniform and dense nanogap distribution because cracks are formed in multiple directions by a non-directional stress distribution.

Through this example, it was confirmed that by controlling the ultrasonic treatment time (tₛₒₙᵢc), the average size of the nanogap, the crack density, and the corresponding sensitivity and detection limit can be quantitatively controlled. This increases the possibility of industrial application in that the performance of the sensor can be adjusted with only one process variable.

### Experimental Example 5: Evaluation of Hydrogen Sensing Characteristics and Selectivity

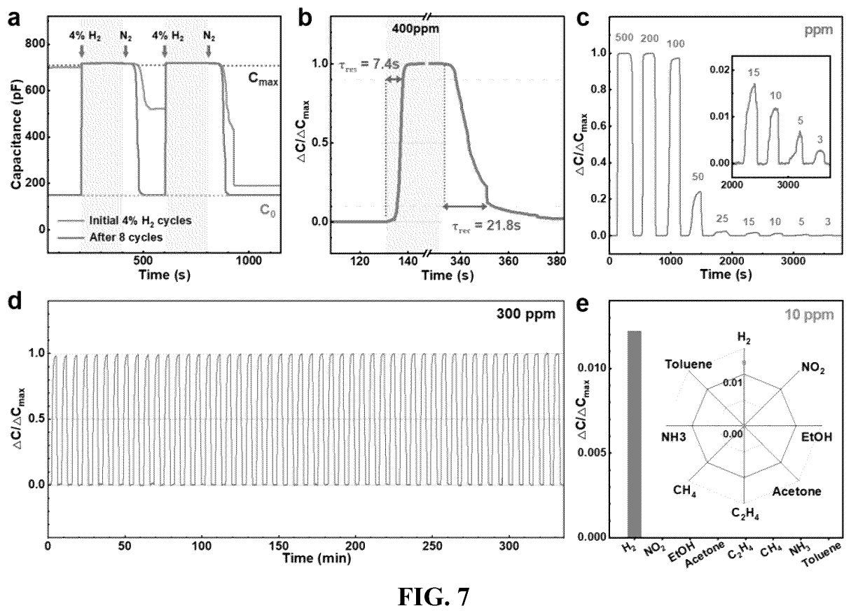

FIG. 7 is a result of quantitatively analyzing the hydrogen sensing performance of the Example 1 sensor, in which sensing characteristics such as response/recovery time, repeatability, and selectivity were evaluated from various angles.

FIG. 7a is the result of repeatedly injecting 4% hydrogen and N₂ cycles to the sensor, and shows that ΔC is stably maintained between the reference capacitance (Cₒ) and the maximum capacitance (Cₘₐₓ). In particular, even after 8 repeated injections, the reference value of the capacitance (Cₒ) was maintained without change, which demonstrated the reproducibility and stability of the sensor structure.

FIG. 7b is the result of measuring a single cycle response when exposed to hydrogen at a concentration of 400 ppm, and the response time was measured as 7.4 seconds and the recovery time as 21.8 seconds. This shows that a fast sensing speed can be secured even under a room temperature (20° C.) condition.

FIG. 7c is a graph measuring the capacitance change (ΔC/ΔCₘₐₓ) of the sensor at various concentrations in the range of 3-500 ppm. Among these, a significant sensing signal was observed even under the 3 ppm condition, experimentally demonstrating that the LOD (limit of detection) of this sensor is at the 3 ppm level.

FIG. 7d is the result of 50 repeated injections under a 300 ppm condition, in which the normalized value of the sensing signal was maintained within a range of ±10% in all cycles, which indicates that the repeatability and durability of the sensor are excellent.

FIG. 7e is a graph comparing the sensing characteristics for various gases as a result of a selectivity evaluation, including H₂ at a concentration of 10 ppm, as well as NO₂, NH₃, CH₄, C₂H₄, EtOH, Acetone, and Toluene. Almost no capacitance change occurred for gases other than H₂, which is determined to be based on the selective hydrogen adsorption mechanism of Pd.

Meanwhile, as a result of quantifying the Average Crack Density (ACD) according to the ultrasonic treatment time through OM image analysis, it was confirmed to be about 130 cracks/mm² under the tₛₒₙᵢc=5 minutes condition, about 80 cracks/mm² at 30 seconds, and about 45 cracks/mm² at 10 seconds.

At this time, the crack density deviation between locations was at the level of ±5, ±10, and ±15, respectively, which indicates that as the ultrasonic treatment time increases, not only the crack density but also the distribution uniformity improves.

This serves as numerical evidence that demonstrates that the process of the present invention is superior in terms of structural precision and reproducibility compared to the conventional MOTIFE method.

Through this example, it could be confirmed that the sensor according to the present invention senses low concentrations of hydrogen quickly and repeatedly, and exhibits high selectivity by not reacting to other gases. These characteristics are important factors that increase the possibility of commercialization as a sensor for real-time hydrogen leakage detection and quantitative analysis.

The foregoing description of the present invention is for exemplary purposes, and it will be understood by those of ordinary skill in the art to which the present invention pertains that various changes in form and details may be made therein without departing from the technical spirit or essential features of the present invention. Therefore, the embodiments described above should be understood in all respects as illustrative and not restrictive. For example, each component described as a single unit may be implemented in a distributed manner, and likewise, components described as distributed may also be implemented in a combined form.

The scope of the present invention is indicated by the claims set forth hereinafter, and all changes or modifications derived from the meaning and scope of the claims and their equivalents should be construed as being included within the scope of the present invention.

What is claimed is:

1. A hydrogen gas measurement sensor, comprising
an insulating substrate;
an oxide layer stacked on the insulating substrate;
a rubber substrate stacked on the oxide layer; and
a catalyst layer stacked on the rubber substrate;
wherein the catalyst layer comprises at least one gap located on a surface or inside thereof.

2. The hydrogen gas measurement sensor of claim 1, wherein the insulating substrate comprises a doped silicon (Si) substrate.

3. The hydrogen gas measurement sensor of claim 1, wherein the oxide layer comprises silicon oxide (SiO₂).

4. The hydrogen gas measurement sensor of claim 1, wherein the rubber substrate comprises polydimethylsiloxane (PDMS).

5. The hydrogen gas measurement sensor of claim 1, wherein the catalyst layer comprises at least one selected from the group consisting of palladium (Pd), platinum (Pt), aluminum (Al), nickel (Ni), manganese (Mn), molybdenum (Mo), magnesium (Mg), vanadium (V), and combinations thereof.

6. The hydrogen gas measurement sensor of claim 1, wherein the gap is an electrical disconnection structure (nanogap) operably formed through a hydrogenation and dehydrogenation process of a microcrack induced by ultrasonic treatment.

7. The hydrogen gas measurement sensor of claim 1, wherein the gap has a width of to 50 nm.

8. The hydrogen gas measurement sensor of claim 1, wherein an interval between the gaps is 0.01 to 5 μm.

9. The hydrogen gas measurement sensor of claim 1, wherein even after repeating adsorption and desorption of hydrogen 50 times or more under a condition of a hydrogen gas concentration of 300 ppm, an opening and closing behavior and a capacitance change of the gap are maintained in a range of 90% to 110% of a reference value.

10. A method for manufacturing a hydrogen gas measurement sensor, comprising the steps of:
(a) forming an insulating substrate;
(b) forming an oxide layer on the insulating substrate;
(c) forming a rubber substrate on the oxide layer;
(d) forming a catalyst layer on the rubber substrate; and
(e) performing an ultrasonic treatment on the catalyst layer to form at least one gap on a surface or inside of the catalyst layer.

11. The method for manufacturing a hydrogen gas measurement sensor of claim 10, wherein the ultrasonic treatment is performed for 5 seconds to 5 minutes in an ethanol solvent.

12. The method for manufacturing a hydrogen gas measurement sensor of claim 10, wherein the gap is an electrical disconnection structure (nanogap) operably formed through a hydrogenation and dehydrogenation process of a microcrack induced by ultrasonic treatment.

13. The method for manufacturing a hydrogen gas measurement sensor of claim 10, further comprising the step of:
(f) performing a hydrogen gas (H₂) treatment.

14. The method for manufacturing a hydrogen gas measurement sensor of claim 13, wherein the hydrogen gas has a concentration of 1 to 10 vol %.

15. The method for manufacturing a hydrogen gas measurement sensor of claim 13, wherein the step (f) is performed by exposure to hydrogen gas for 1 minute to 5 minutes.
